(19) United States
(12) Patent Application Publication
(10) Pub. No.: US 2025/0256098 A1
(43) Pub. Date: Aug. 14, 2025

(54) DEVICE AND METHOD FOR OBTAINING SHORT-LATENCY SEPS USING EAR-PLACED ELECTRODES

(71) Applicant: Alphatec Spine, Inc., Carlsbad, CA (US)

(72) Inventors: Richard Arthur O'Brien, Cockeysville, MD (US); Robert Gerard Snow, Phoenix, MD (US)

(21) Appl. No.: 19/049,911

(22) Filed: Feb. 10, 2025

Related U.S. Application Data
(60) Provisional application No. 63/553,306, filed on Feb. 14, 2024.

(51) Int. Cl. A61B 5/377; A61B 5/372; A61B 5/384

(52) U.S. Cl. CPC A61N 1/36036; A61B 5/372; A61B 5/377; A61B 5/384

(57) ABSTRACT

Disclosed herein are methods, devices, and systems for obtaining, sometimes in an automated manner, from a patient a combination of subcortical and cortical somatosensory evoked response potentials, EEG, and Brainstem auditory evoked responses using ear electrodes and surface electrodes. These methods, devices, and systems may be employed during a surgical procedure in conjunction with or separate from other monitoring modalities to monitor the patient's health generally and/or with respect to specific aspects of the patient's health prior to, during, and/or after the surgical procedure.

### RELATED APPLICATIONS

The present application claims priority from U.S. Provisional Application No. 63/553,306, filed Feb. 14, 2024, the entirety of which is incorporated herein by reference.

### BACKGROUND

The present disclosure relates generally to neurophysiological monitoring in the operating room in which anesthesia is applied and/or where the nervous system may be at risk.

U.S. Publication No. 2006/0167722 discusses a system for determining and maintaining a concentration level of medication in a patient sufficient to achieve and maintain a desired effect on that patient. Generally speaking, a medication delivery controller uses a patient response profile to determine a concentration of medication in the patient that will achieve the desired effect on the patient. The patient response profile is a graphical, tabular, or analytical expression of the relationship between the concentration of a medication and the effect of the medication at the specific concentration. Using this information, the medication delivery controller provides instructions to a medication delivery unit such as, for example, an infusion pump or inhalation device, to deliver the medication to the patient at a rate that will achieve the desired concentration level of the medication in the patient.

U.S. Pat. No. 4,907,597 discusses a biopotential analysis system for determining, in a noninvasive manner, cerebral electrical properties. A suitable electrode and amplifier system is used to obtain biopotentials from the region of interest. Surface electroencephalographic signals are processed in various ways to display a bicoherence array on a video terminal or to use the same as a figure of merit for the assessment of cerebral electrical function.

Kidmose et al. performed a rigorous comparison between in-ear EEG monitoring and conventional on-scalp EEG for both auditory and visual evoked responses, over steady-state and transient paradigms, and across a population of subjects. The outcomes of this study demonstrated that the ear-EEG signals, in terms of the signal-to-noise ratio, are on par with conventional EEG recorded from electrodes placed over the temporal region. P. Kidmose et al., A Study of Evoked Potentials From Ear-EEG, 60(10) IEEE TRANS. BIO. ENG. 2824-30 (Oct. 2013).

Meiser et al. highlight some of the advantages of ear-EEG over classical cap-EEG particularly the ability to collect physiological recordings in everyday life away from a lab, though such mobility comes with the limitations of not being able to collect as much data as traditional methods. A. Meiser et al., Ear-EEG compares well to cap-EEG in recording auditory ERPs: a quantification of signal loss, 19 J. NEURAL ENG. (2022).

Similar to Meiser et al., Kidmose et al. recognized the flexibility that comes with in-car EEG monitoring and compared Ear-EEG responses with conventional on-scalp recordings and with well-established results from the literature. Both steady state and transient responses can be obtained from Ear-EEG, and these responses have similar characteristics and quality compared to EEG obtained from conventional on-scalp recordings. P. Kidmose et al., Auditory Evoked Responses from Ear-EEG Recordings, 34TH ANNUAL INT'L CONF. IEEE EMBS 586-89 (28 August-1 September 2012).

Kuatsjah et al. studied the correlation between in-ear EEG monitoring and the stress experienced by a subject when performing visuomotor tasks showing that such monitoring can be effective to detect the level mental workload experienced by the subjects. E. Kuatsjah et al., Two-channel in-ear EEG system for detection of visuomotor tracking state: A preliminary study, 68 MED. ENG. & PHYS. 25-34 (June 2019).

Hwang et al. evaluated the performance of in-car EEG in drivers' alertness-drowsiness classification and tested three peripheral signals: electrocardiogram (ECG), photoplethysmogram (PPG), and galvanic skin response (GSR). The classification analysis using the in-ear EEG resulted in high classification accuracy comparable to that of the individual on-scalp EEG channels. The ECG, PPG, and GSR showed competitive performance but only when used together in pairwise combinations. T. Hwang et al., Driver drowsiness detection using the in-ear EEG, 38TH ANNUAL INT'L CONF. IEEE EMBC (26-20 August 2016).

During surgical procedures requiring anesthesia patients may be monitored using neurophysiological techniques and parameters to detect early changes in neurological function that if left un-mitigated might lead to transient or permanent injury or dysfunction post operatively (Intra-Operative Neurological Monitoring or IONM). Such monitoring may use a wide range of techniques including for example Short latency Somatosensory Evoked Potentials (SSEP), Trans Cortical Electric Motor Evoked Potentials (TceMEP), Electroencephalography (EEG), Hoffman waves (H waves), spontaneous electromyography (sEMG), near nerve stimulation and recording from nerve and/or muscle (triggered EMG) and neuromuscular junction testing for paralytic effect (e.g. Train of Four (TOF) stimulation and muscle recording).

In addition, anesthesiologists may monitor depth of anesthesia utilizing a variety of techniques such as Bispectral Index (BIS) (see U.S. Pat. No. 5,320,109). As many anesthetic agents affect the quality and reliably of the intraoperative monitoring signal that can be obtained, such information is useful to both the anesthesiologist and the intraoperative monitoring team.

IONM is currently used in a variety of surgical procedures, most notably spinal surgeries, which account for 70-80% of surgical cases that utilize IONM. In spinal surgery IONM, the focus is on the techniques that measure the spinal elements along the course of the spinal cord that are at risk including SSEP, TceMEP and sEMG.

While current intraoperative monitoring devices and techniques provide useful information for the surgical team, they suffer from several problems. Both SSEPs and TceMEPs are subject to significant degradation from anesthetic agents, particularly halogenated gases, often requiring use of more expensive total intravenous anesthesia (TIVA) using drugs such a propofol. Additionally, there is no easy and automated way to integrate information that is not specifically collected by the IONM devices that might cause signal change when considering whether to alert a user to undesirable changes. The monitoring professional must therefore subjectively integrate these events, including changes in blood pressure, perfusion, anesthetic depth, and temperature.

In addition, techniques like SSEP and TceMEP typically utilize subdermal needle electrodes that risk local tissue injury, bleeding, and needlestick injuries. Accurate placement of these electrodes is required for adequate monitoring and typically requires a trained individual often overseen by a physician other than the surgeon.

EEG recorded during surgeries utilizes multiple needle electrodes over the scalp or can be recorded directly from the brain using specialized grid electrodes for select cranial surgeries. Ear-inserted electrodes may also be used to obtain auditory evoked potentials in response to auditory click stimuli (Kidmose 2012) in both the diagnostic setting and during cranial surgeries.

Outside the operating room, EEG is used in diagnostic settings with application of a large array of eight or more electrodes. More recently there is considerable interest in ambulatory recording of EEG potentials in the wearable technology industry space by placing specialized multi-lead electrodes in the external ears (so called ear-EEG) (see U.S. Pat. No. 5,320,109). This interest arises from the circumstance that the external and internal auditory meatuses penetrate the boney skull and provide ear electrodes with a ‘keyhole’ for viewing cortical potentials. Several possible ambulatory applications have been explored like sleep and drowsiness monitoring, relaxation therapy, speech imagery, hypoglycemia detection, hearing aid enhancement and device remote control. Some authors have also tested ear electrodes for viewing other supra-tentorial generated potentials such as visual evoked potentials and eye movement potentials for visual tracking.

### SUMMARY

While all these techniques are of interest, neither standard multiple needle electrode recorded EEG in the operating room nor ambulatory ear electrode recording techniques have significant application in spine surgery IONM as they do not monitor the spinal elements along the course of the spinal cord at risk.

What is needed is an automated, less disruptive method and device for recording spinal elements along the course of the spinal cord that are at risk, that does not involve needle electrodes, does not require TIVA, can be easily and simply applied without expert attendance, and can be automatically integrated with measurements of anesthetic effect to help prevent false positive monitoring alerts.

In an exemplary embodiment of the present invention a system, method, device, apparatus, and/or computer program product for automatically detecting and identifying impending neural injury and anesthetic levels during spinal surgery using ear electrodes and surface electrodes is disclosed.

According to an exemplary embodiment of the invention a method of identifying neural injury and anesthetic change during surgery is provided.

According to an exemplary embodiment, electrical waveforms representing electroencephalography, brainstem auditory evoked potentials, and somatosensory evoked potentials may be collected.

According to an exemplary embodiment, electrodes may be attached, coupled, and/or connected to the patient with stimulating electrodes that include surface electrodes over one or more peripheral nerves in one or more extremities.

According to an exemplary embodiment, recording electrodes may be attached, connected, and/or coupled over one or more parts of one or both external cars and car canals and, optionally, on one or more areas of the forehead.

According to an exemplary embodiment stimulation of hearing may be delivered through the car electrodes.

According to an exemplary embodiment stimulation of one or more extremity nerves occurs sequentially or intermittently or by pre-designated sequence.

According to an exemplary embodiment auditory stimulation of one or more cars occurs sequentially or intermittently or by pre-designated sequence.

According to an exemplary embodiment recording of brainstem and cerebral waveforms occurs through one or more surfaces embedded in the car electrodes and, optionally, through surface electrodes on one or more areas of the forehead.

According to an exemplary embodiment, by use of the computing device, various aspects of a stimulation may be automatically controlled including but not limited to intensity, stimulus duration, frequency, stimulation sequence, and recording duration.

According to an exemplary embodiment, identifying changes in neural function may include one or more of comparing, with the computing device, information based on the resultant electrical waveforms obtained at the beginning of the surgical case. Such waveforms may be characterized as baselines to be compared to those obtained later in the surgical case using one or more parameters including but not limited to amplitude, latency, morphology, and area under the curve using an algorithm or artificial intelligence method.

According to an exemplary embodiment, the identifying of changes in neural function may include one or more of comparing, with the computing device, information based on the resultant electrical waveforms generated from one or more portions of the nervous system over time, including information indicating anesthesia level calculated, with the one or more computing devices, from the same electrical waveforms, to determine when changes in the resultant electrical waveforms are due to anesthesia or some other cause or both using an algorithm or artificial intelligence method.

The method may further include displaying information based on the resultant electrical waveforms on a display unit.

According to an exemplary embodiment, the method may further include alerting a user to changes in the resultant waveforms using one or more of a notification, an alert, a communication, an indication, and/or an alarm.

In an exemplary embodiment, the device may include a processor, a memory, a storage device, or a computer.

According to an exemplary embodiment, the response to several stimuli may be averaged together to reduce noise and produce a clean signal. In an exemplary embodiment, proprietary or third-party software may be used in signal processing to improve the signal-to-noise ratio.

In an exemplary embodiment, the alert and display unit may include a display which may display various information, such as, e.g., but not limited to, areas being stimulated and recorded from, baseline and current signal traces, trends in signals, relevant changes in signals, location of signal changes, quality of recorded signals, position of electrodes, and alerts due to significant changes in signal. In addition, the alert and display unit may include multiple buttons or control inputs.

According to an exemplary embodiment, the buttons or inputs may allow an operator to set up the initial monitoring layout and interact with the alert and display unit during monitoring to add additional information or respond to alerts. In an exemplary embodiment, the alert and display unit may allow override of a change in signal by an anesthesiologist, or other medical personnel, etc. when a signal change is related to a change in dose of paralytic agent or some other event unrelated to nerve injury.

The present embodiments (or any part(s) or function(s) thereof) may be implemented using hardware, software, firmware, or a combination thereof and may be implemented in one or more computer systems or other processing systems. In fact, in one exemplary embodiment, the invention may be directed toward one or more computer systems capable of carrying out the functionality described herein.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be better understood when read in conjunction with the following drawings wherein like structure is indicated with like reference numerals and in which:

FIG. 1 illustrates an exemplary embodiment for the placement of electrodes on a patient according to the present disclosure.

FIG. 2 illustrates an electrical somatosensory evoked response waveform recorded from an exemplary embodiment according to the present disclosure.

### DETAILED DESCRIPTION

The present disclosure relates to physiological monitoring systems and methods that utilize electrodes placed on or in a patient's ear.

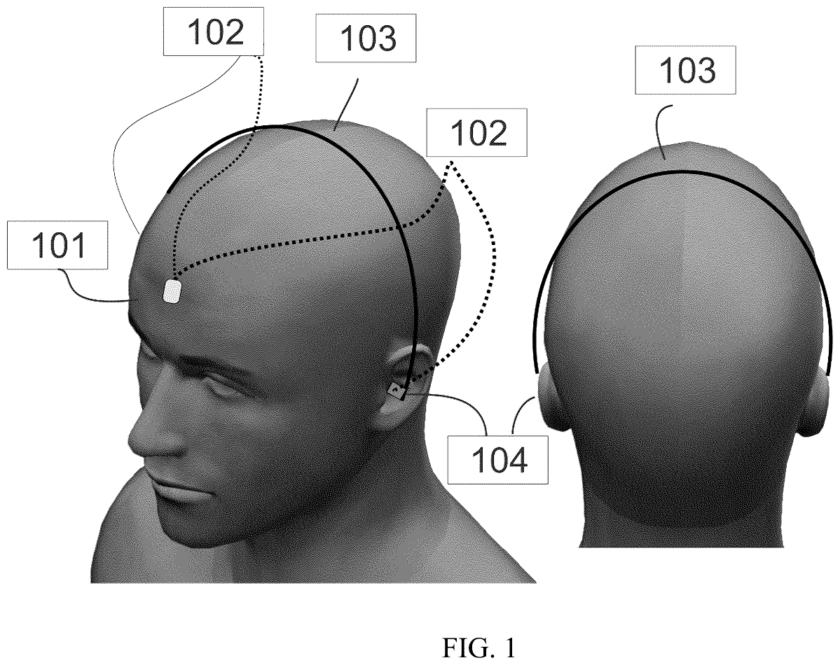

FIG. 1 shows an exemplary embodiment of the placement of a forehead electrode (101) and ear electrodes (104) arranged in a recording method (102) utilizing a band device (103) to hold them in position. The illustrated embodiment utilizes at least one electrode 104 placed in or on the ear with another electrode placed in the opposite ear or, alternatively, at least one forehead electrode 101 placed on at least a portion of the forehead.

According to some embodiments, the electrodes identified in FIG. 1 are placed on a patient prior to a surgical procedure. They may be placed by a trained professional or any member of the medical staff attending the surgery. Little or no training may be required to place the electrodes.

Though not illustrated, exemplary embodiments may further include electrodes (recording and/or stimulating electrodes) positioned on one or more peripheral nerves of the patient.

The electrodes are connected to, or may already be in communication with (wirelessly or through a wired connection), a stimulation and monitoring system that may be configured to operate automatically or may be configured to accept user input, which input may include instructions to begin monitoring, after which all monitoring and stimulation are controlled by the system as it operates autonomously. In some embodiments, input is accepted during the monitoring and stimulation process.

According to one understanding, an advantage of the presently disclosed methods, apparatuses, and systems is the ability to monitor a patient's condition by using not just one nerve pathway (e.g., sensory or auditory) but two (i.e., sensory and auditory). By utilizing both pathways, it is believed that better monitoring can be achieved and/or that neural monitoring is made to be less invasive, more predictable, and/or requiring less skilled training to implement.

An exemplary method of monitoring and stimulation is implemented by one or more processors contained with the system that have stored therein instructions to automatically obtain from a patient a combination of subcortical and cortical somatosensory evoked response potentials. Some embodiments are further configured to obtain an EEG signal from the patient. Some embodiments are further configured to obtain brainstem auditory evoked responses. Some embodiments are employed during a surgical procedure though may be initiated just prior to the procedure in order to obtain baseline responses.

Some systems according to the present disclosure are configured to provide stimulation via an electrode positioned over at least one peripheral nerve of the patient. The electrode may be placed on at least one extremity of the patient. The electrode may include a single electrode or multiple electrodes configured to operate in unison. Such systems are further configured to provide auditory stimulation to at least one ear of the patient. The systems further include one or more recording electrodes positioned over or within in at least one external car canal as well as, optionally, one or more recording electrodes positioned on at least a portion of the patient's forehead.

The system is configured to detect, using electrical signals from the recording electrode(s) electrical responses from the brainstem and/or cortex. Some systems may also record spontaneous electrical waveforms from the cerebrum. The system then records the electrical signals, or electrical waveforms, that are either spontaneously produced or that result from stimulations (electrical and/or auditory) provided by the system. The one or more processors of the system are configured to average the electrical waveforms resulting in response to the electrical and auditory stimulation and to detect automatically the initial state or baseline of the electrical signals, thereby establishing the patient's initial state prior to or during the surgical procedure.

Some systems are configured to provide further stimulations after a baseline has been established or even absent a baseline being established. The further stimulation may be initiated by a user at any time during the surgical procedure, or it may be initiated automatically by the system either on a pre-determined interval—which may be established by the user in advance and/or adjusted during surgery—or as determined by the system based on the resulting electrical waveforms or on a particular stage of the surgery. For example, if the system determines that the resulting electrical waveforms are indicative of a potential problem, such as a risk to neural function, the system may implement additional stimulation or a greater amount of stimulation to determine if the potential problem is an actual problem and/or if an alert should be generated so as to attract the user's attention or to perform some of ameliorative action. In some embodiments, the system is configured to accept as inputs information indicative of a stage of the surgical procedure where certain stages are associated with greater risk to the patient. With such information, the system may implement additional stimulations and/or a greater number of stimulations over a given period to provide greater confidence in the monitoring during those stages of greater risk to the patient.

The systems are configured to detect a change in the resultant electrical waveforms that may be indicative of a risk to the patient or damage that may have been caused to the patient, such as a change in neural function, positioning effect, or nerve damage caused by the procedure or the instrumentation. The system analyzes the detected changes, comparing the changes to a baseline and/or to prior resulting electrical waveforms. In some systems, the resultant electrical waveforms are averaged together to reduce the impact of unwanted noise. To measure such changes, the system may monitor a latency, an amplitude, a frequency content, and/or morphology of the resulting electrical waveforms. In some embodiments, the electrical waveforms are muscle waveforms.

The injury or damage to the patient may be along a neural pathway associated with a particular surgical procedure. The resultant electrical waveforms may originate from the patient's brainstem or the cerebrum, or both. In some embodiments, the system is configured to compare signals between different limbs of the patient.

The detected changes may also or alternatively be indicative of a change in the level of anesthesia in the patient. Some systems of the present disclosure are configured to identify whether such detected changes are the result of anesthesia changes or risks to the patient or both. In some embodiments, the determination of whether detected changes are the result of anesthesia is accomplished using an algorithm, while in some embodiments, the determination is made using an artificial intelligence classifier.

According to some embodiments, the monitoring systems disclosed herein are in communication with other monitoring systems in the operating room, such as, but not limited to, an anesthesia machine, a blood pressure monitor, a pulse oximetry machine, a neuromuscular junction testing machine. Such machines or monitors may provide to the system information on their operation and/or physiological data on the patient, which information may be incorporated by the system in its analysis and determination as to whether the patient has or is suffering from a neural injury resulting from the surgical procedure.

Some systems are configured to provide up-to-date information to a display visible to a user. Such information may include the patient's current health, the current level of anesthesia, or both. When a change is detected in the resulting electrical waveforms, the information may include such changes and whether the changes are determined to be harmful or not and whether the user should take action. In some embodiments, the system may provide an alert, such as a visual alert on the display or an audible alert to the user so that a problem can be ameliorated. In some cases, the problem may be addressed by moving the patient. In some cases, the problem may be addressed by halting or modifying the surgery. The alert provided to the user may come in the form of an alarm, a verbal communication, or some other notification. In some embodiments, the screen may provide the first indication of a problem, potential or otherwise, and then an audible alert may sound if no improvement is detected (or if the user does not acknowledge the first indication), and then a louder alarm may sound if still no improvement is detected (or if the user does not acknowledge the audible alert). In some embodiments, the audible alert may begin at a relatively low volume and increase until the system determines that the risk has been addressed and/or until the user acknowledges the alert.

The up-to-date information and/or alerts may include a visual representation of the patient's body identifying portions of the body that, according to the system, are functioning properly and/or that are not functioning properly perhaps because of an injury or an issue with a neural pathway.

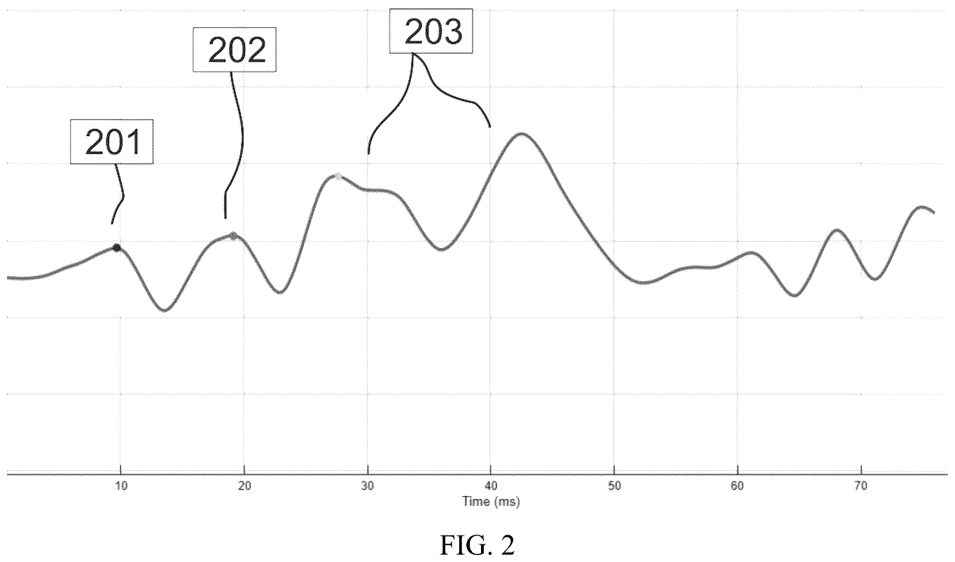

FIG. 2 shows an exemplary embodiment of an electrical somatosensory evoked response waveform recorded from one or more of a patient's ears car and, optionally, on the patient's forehead electrodes containing subcortical (201), cortical (202) and late cortical (203) components. The displays discussed herein may provide such graphical information in addition to or instead of a representation of the patient's body.

In some situations, the surgical procedure is a spinal procedure, such as ACDF, PCF, TLIF, LIF, PTP, ALIF, GLIF, or PLIF. In some situations, the surgical procedure that is performed while the patient is unconscious. The systems disclosed herein are designed to provide a user with at least some information that could be provided by the patient if the patient were awake and responsive or if the patient had fully sensory perception of the areas of their body being operated on.

In some embodiments, the system is configured to provide auditory stimulation periodically on a predetermined basis while also providing stimulation to a peripheral nerve on the same or a different schedule.

According to some embodiments, the system is configured to determine through ongoing stimulation of the ear and/or peripheral nerves whether the ongoing stimulation is adequate, meaning that the system has a reasonable level of confidence in the results being provided to the display. In some embodiments, the system may provide an indication of its confidence in the information, such as by indicating a percentage value or a color indication of the confidence level, or a combination of the two. For example, a relatively high confidence rating may be shown in green text while a relatively low confidence rating may be shown in red text.

In some embodiments, the system may prompt the user to accept the data provided to the user thereby indicating to the system that the data can be considered accurate so as to allow the system to continue monitoring. In some embodiments, the user may indicate that the data provided is not accurate, which may cause the system to automatically implement one or more modifications to the stimulations so as to improve their accuracy. In some embodiments, the system may propose modifications to the stimulations, which the user must accept or reject before the system continues with any stimulations.

According to some embodiments, the system is configured to determine through ongoing stimulation and monitoring whether modifications to the stimulations may elicit improved responses. For example, the system may make minor adjustments to duration, stimulation level, periodicity, frequency, correlation between the various stimulation electrodes, etc. of the stimulations and monitoring the resulting electrical waveforms and make a determination as to whether any aspects of the stimulations need to be adjusted to provide more reliable results. In some embodiments, the system is configured to automatically make such a determination and to automatically implement that determination. In some embodiments, the system prompts the user to report the system's findings that one or more changes in the stimulations could provide better results and asks whether the user would like the system to implement the change(s).

### Embodiments

The following embodiments are provided as examples only of specific configurations, materials, arrangements, etc. contemplated by the authors of this disclosure:
Embodiment 1. A method of automatically obtaining from a patient a combination of subcortical and cortical somatosensory evoked response potentials, EEG, and Brainstem auditory evoked responses during a surgical procedure using ear electrodes and surface electrodes, the method implemented automatically by a computing device, the method comprising:
   providing at least one of electrical stimulation via a first electrode positioned over at least one peripheral nerve of at least one extremity of the patient and auditory stimulation via a second electrode positioned over or within a first ear of the patient recording via a third electrode associated with the second electrode over or within the first ear of the patient, the third electrode utilizing a fourth reference electrode;
   detecting electrical responses from the brainstem and cortex;
   recording electrical waveforms produced both spontaneously and those resulting from the electrical and auditory stimulation;
   averaging the electrical waveforms resulting from the electrical and/or auditory stimulation;
   automatically detecting an initial state of the electrical responses;
   further stimulating the first and/or second electrodes;
   detecting a change in the resultant electrical waveforms during further recording over time;
   analyzing the detected change in the resultant electrical waveforms over time to automatically identify injury or potential injury along the associated neural pathways;
   analyzing the detected change in the resultant electrical waveforms to automatically identify changes in anesthesia level over time;
   integrating the combined available information from the electrical waveforms to determine if the detected change in the electrical waveforms is due to anesthesia or potential injury or both;
   forwarding data to a display, the data comprising information for displaying the waveforms or an anatomical diagram that depicts the location of the detected change.
Embodiment 2. The method of embodiment 1, wherein the fourth reference electrode is placed on or near the patient's forehead.
Embodiment 3. The method of embodiment 1, wherein the fourth reference electrode is placed over or inside a second ear of the patient.
Embodiment 4. The method of any one of embodiments 1, 2 or 3, wherein the surgical procedure is a spinal surgery.
Embodiment 5. The method of any one of embodiments 1, 2, 3, or 4, wherein the auditory stimulation is periodic.
Embodiment 6. The method of any one of embodiments 1, 2, 3, 4, or 5, wherein said at least one recording electrode is coupled to at least one ear and one part of the forehead.
Embodiment 7. The method of any one of embodiments 1, 2, 3, 4, 5, or 6, wherein the resultant electrical waveforms are responses from either the brainstem or cerebrum, or both.
Embodiment 8. The method of any one of embodiments 1, 2, 3, 4, 5, 6, or 7, wherein automatically determining, if the resultant electrical waveforms are adequate for ongoing monitoring includes the results of further stimulation.
Embodiment 9. The method of any one of embodiments 1, 2, 3, 4, 5, 6, 7, or 8, further comprising determining if the resultant electrical waveforms might be improved by a change in the stimulation frequency, intensity, or duration.
Embodiment 10. The method of any one of embodiments 1, 2, 3, 4, 5, 6, 7, 8, or 9, wherein integrating the combined available information from the electrical waveforms is performed by an algorithm or by an artificial intelligence classifier.
Embodiment 11. The method of any one of embodiments 1, 2, 3, 4, 5, 6, 7, 8, 9, or 10, further comprising alerting a user to the detected change using at least one of:
   a notification;
   an alert;
   a communication;
   an indication; or
   an alarm.
Embodiment 12. The method of any one of embodiments 1, 2, 3, 4, 5, 6, 7, 8, 9, 10, or 11, further comprising displaying information based on the resultant electrical waveforms on at least one display.
Embodiment 13. The method of any one of embodiments 1, 2, 3, 4, 5, 6, 7, 8, 9, 10, 11, or 12, further comprising receiving a user input regarding the accuracy of the resultant electrical waveforms.
Embodiment 14. The method of any one of embodiments 1, 2, 3, 4, 5, 6, 7, 8, 9, 10, 11, 12, or 13, wherein the further stimulating step is due to user intervention, a predetermined frequency or sequence, or in response to a detected change in the electrical signals.
Embodiment 15. An automated apparatus for detecting neural dysfunction and anesthesia effect during spinal surgery on a patient, the apparatus comprising:
   an output operable to couple to at least one of at least one stimulating electrode to stimulate one or more nerves or one or more extremities of the patient and at least one stimulating electrode to stimulate one or more ears of the patient with an auditory signal;
   an input operable to couple to at least one recording electrode to record resultant electrical waveforms generated by the patient's brainstem and/or cerebrum to the one or more stimulating electrodes;
   at least one processor, coupled to said output and said input, operable to:
   detect a change in the resultant electrical waveforms, wherein the detected change in the resultant electrical waveforms indicates a change in neural function or a change in anesthesia level or both, the change in neural function comprising an immediate or potential injury to the one or more peripheral nerves or conducting spinal elements due to stress associated with a surgical occurrence during the surgery;
   and a display interface operable to receive and display data from the at least one processor, the data comprising information for displaying an anatomical diagram that depicts a location of the detected change.
Embodiment 16. The apparatus of embodiment 15, further comprising an input operably connected to at least one recording electrode to record resultant electrical waveforms generated spontaneously by the cerebrum through the auditory pathway.
Embodiment 17. The apparatus of embodiment 15 or 16, wherein the change in the resultant electrical waveforms comprises a change in one or more of a latency, amplitude, frequency content, or morphology of the resultant electrical waveforms.
Embodiment 18. The apparatus of any one of embodiments 15, 16, or 17, further comprising an alert unit coupled to said at least one processor operable to alert a user to the detected change.
Embodiment 19. The apparatus of any one of embodiments 15, 16, 17, or 18, further comprising at least one of: means for obtaining information from an anesthesia machine to determine when changes in the resultant electrical waveforms are due to anesthesia; an anesthesia machine; a blood pressure machine; or means for obtaining information from a blood pressure machine to determine when changes in the resultant electrical waveforms are due to blood pressure; a pulse oximetry machine; or means for obtaining information from a pulse oximetry machine to determine when changes in the resultant electrical waveforms are due to a change in perfusion; a neuromuscular junction testing machine; or means for obtaining information from a neuromuscular junction testing machine to determine when changes in the resultant electrical waveforms are due to a change in paralytic action.
Embodiment 20. The apparatus of embodiment 19, wherein the resultant electrical waveforms are muscle waveforms.
Embodiment 21. The apparatus of embodiment 19 or 20, wherein the at least one processor is operable to generate a recommendation for action to mitigate the identified neural dysfunction.
Embodiment 22. The apparatus of any one of embodiments 19, 20, or 21, wherein the at least one processor is further operable to average together resultant electrical waveforms generated in response to several stimulations to reduce noise.
Embodiment 23. The apparatus of any one of embodiments 19, 20, 21, or 22, wherein the at least one processor is further operable to compare signals between different limbs of a patient.
Embodiment 24. The apparatus of any one of embodiments 19, 20, 21, 22, or 23, wherein the apparatus comprises a plurality of stimulation outputs and a plurality of recording inputs.
Embodiment 25. A system comprising:
   the apparatus of any one of embodiments 16, 17, 18, 19, 20, 21, 22, 23, or 24;
   one or more recording electrodes electrically coupled to the apparatus; and one or more stimulating electrodes electrically coupled to the apparatus.
Embodiment 26. The system of embodiment 25, further comprising an output unit mechanically, electrically, or electronically coupled to the apparatus.
Embodiment 27. A method of identifying neural dysfunction and anesthetic effect using ear and forehead electrodes in a surgical patient, the method comprising:
   stimulating at least one of one or more peripheral nerves in one or more extremity and one or more ears with sound;
   recording resultant electrical waveforms generated by the brainstem and cerebrum in response to the stimulations using a recording electrode in one ear with a reference electrode in the other ear or over one or more areas of the forehead;
   recording spontaneous electrical waveforms from the cerebrum using a recording electrode in one ear with a reference electrode in the other ear or over one or more areas of the forehead;
   detecting, by a computing device, by various means, a change in the resultant electrical waveforms;
   analyzing, by the computing device, by various means, the detected change in the resultant electrical waveforms to automatically identify the neural dysfunction, the neural dysfunction comprising an immediate or potential injury to the one or more nerve structures due to stress associated with a surgical occurrence during a surgery;
   analyzing, by the computing device, by various means, the detected change in the resultant electrical waveforms to automatically identify changes in anesthesia during a surgery;
   analyzing, by the computing device, by various means, the detected change in the resultant electrical waveforms to automatically identify the effects of anesthesia on all of the resultant electrical waveforms;
   and forwarding data to a display, the data comprising at least one of the electrical waveforms and an anatomical or functional diagram that depicts a location or degree of the detected change.
Embodiment 28. An apparatus for automatically detecting neural dysfunction and anesthesia effect during a surgical procedure on a patient, the apparatus comprising:
   a first electrode configured to provide electrical stimulation to a sensory nerve of the patient;
   a second electrode configured to provide auditory stimulation to an auditory nerve of the patient;
   a third electrode associated with the second electrode, the third electrode configured record resultant electrical waveforms generated by the patient's brainstem and/or cerebrum to the first and second electrodes;
   a fourth electrode configured to serve as a reference electrode for the third electrode;
   at least one processor, coupled to said output and said input, operable to:
      detect a change in the resultant electrical waveforms, wherein the detected change in the resultant electrical waveforms indicates a change in neural function or a change in anesthesia level or both, the change in neural function comprising an immediate or potential injury to the one or more peripheral nerves or conducting spinal elements due to stress associated with a surgical occurrence during the surgery;
      and a display interface operable to receive and display data from the at least one processor, the data comprising information for displaying an anatomical diagram that depicts a location of the detected change.
Embodiment 29. The apparatus of embodiment 28, wherein the first electrode is configured to be positioned over or near a peripheral nerve of the patient.
Embodiment 30. The apparatus of embodiment 28 or 29, wherein the first electrode comprises one or more electrodes.
Embodiment 31. The apparatus of any one of embodiments 28, 29, or 30, wherein the second electrode is configured to be positioned over or at least partially inside a first ear of the patient.
Embodiment 32. The apparatus of any one of embodiments 28, 29, 30, or 31, wherein the third electrode is configured to be positioned over or at least partially inside a first ear of the patient.
Embodiment 33. The apparatus of any one of embodiments 28, 29, 30, 31, or 32, wherein the fourth electrode is configured to be positioned over or at least partially inside a second ear of the patient.
Embodiment 34. The apparatus of any one of embodiments 28, 29, 30, 31, or 32, wherein the fourth electrode is configured to be positioned over or near the patient's forehead.
Embodiment 35. The apparatus of any one of embodiments 28, 29, 30, 31, 32, 33, or 34, wherein the change in the resultant electrical waveforms comprises a change in one or more of a latency, amplitude, frequency content, or morphology of the resultant electrical waveforms.
Embodiment 36. The apparatus of any one of embodiments 28, 29, 30, 31, 32, 33, 34, or 35, further comprising an alert unit coupled to said at least one processor operable to alert a user to the detected change.
Embodiment 37. The apparatus of any one of embodiments 28, 29, 30, 31, 32, 33, 34, 35, or 36, further comprising at least one of: means for obtaining information from an anesthesia machine to determine when changes in the resultant electrical waveforms are due to anesthesia; an anesthesia machine; a blood pressure machine; or means for obtaining information from a blood pressure machine to determine when changes in the resultant electrical waveforms are due to blood pressure; a pulse oximetry machine; or means for obtaining information from a pulse oximetry machine to determine when changes in the resultant electrical waveforms are due to a change in perfusion; a neuromuscular junction testing machine; or means for obtaining information from a neuromuscular junction testing machine to determine when changes in the resultant electrical waveforms are due to a change in paralytic action.
Embodiment 38. The apparatus of embodiment 37, wherein the at least one processor is operable to generate a recommendation for action to mitigate the identified neural dysfunction.
Embodiment 39. The apparatus of embodiments 37 or 38, wherein the at least one processor is further operable to average together resultant electrical waveforms generated in response to several stimulations to reduce noise.
Embodiment 40. The apparatus of any one of embodiments 37, 38, or 39, wherein the at least one processor is further operable to compare signals between different limbs of a patient.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It should also be noted that some of the embodiments disclosed herein may have been disclosed in relation to a particular approach (e.g., lateral); however, other approaches (e.g., anterior, posterior, transforaminal, etc.) are also contemplated.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the embodiments of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements. In one embodiment, the terms “about” and “approximately” refer to numerical parameters within 10% of the indicated range.

The terms “a,” “an,” “the,” and similar referents used in the context of describing the embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the embodiments of the present disclosure and does not pose a limitation on the scope of the present disclosure. No language in the specification should be construed as indicating any non-claimed element essential to the practice of the embodiments of the present disclosure.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the author(s) of this disclosure for carrying out the embodiments disclosed herein. Of course, variations on these described embodiments will become apparent to those of ordinary skill in the art upon reading the foregoing description. The author(s) expects skilled artisans to employ such variations as appropriate, and the author(s) intends for the embodiments of the present disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of this disclosure so claimed are inherently or expressly described and enabled herein.

Furthermore, if any references have been made to patents and printed publications throughout this disclosure, each of these references and printed publications are individually incorporated herein by reference in their entirety.

In closing, it is to be understood that the embodiments disclosed herein are illustrative of the principles of the present disclosure. Other modifications that may be employed are within the scope of this disclosure. Thus, by way of example, but not of limitation, alternative configurations of the embodiments of the present disclosure may be utilized in accordance with the teachings herein. Accordingly, the present disclosure is not limited to that precisely as shown and described.

1. A method of automatically obtaining from a patient a combination of subcortical and cortical somatosensory evoked response potentials, EEG, and Brainstem auditory evoked responses during a surgical procedure using ear electrodes and surface electrodes, the method implemented automatically by a computing device, the method comprising:
providing at least one of electrical stimulation via a first electrode positioned over at least one peripheral nerve of at least one extremity of the patient and auditory stimulation via a second electrode positioned over or within a first ear of the patient
recording via a third recording electrode associated with the second electrode over or within the first ear of the patient, the third electrode utilizing a fourth reference electrode;
detecting electrical responses from the brainstem and cortex;
recording electrical waveforms produced both spontaneously and those resulting from the electrical and auditory stimulation;
averaging the electrical waveforms resulting from the electrical and/or auditory stimulation;
automatically detecting an initial state of the electrical responses;
further stimulating the first and/or second electrodes;
detecting a change in the resultant electrical waveforms during further recording over time;
analyzing the detected change in the resultant electrical waveforms over time to automatically identify injury or potential injury along associated neural pathways;
analyzing the detected change in the resultant electrical waveforms to automatically identify changes in anesthesia level over time;
integrating available information from the electrical waveforms to determine if the detected change in the electrical waveforms is due to anesthesia or potential injury or both;
forwarding data to a display, the data comprising information for displaying the waveforms or an anatomical diagram that depicts a location of the detected change.

2. The method of claim 1, wherein the fourth reference electrode is placed on or near a patient's forehead.

3. The method of claim 1, wherein the fourth reference electrode is placed over or inside a second ear of the patient.

4. The method of claim 1, wherein the surgical procedure is a spinal surgery.

5. The method of claim 1, wherein the auditory stimulation is periodic.

6. The method of claim 1, wherein said recording electrode is coupled to at least one ear and one part of a patient's forehead.

7. The method of claim 1, wherein the resultant electrical waveforms are responses from either the brainstem or cerebrum, or both.

8. The method of claim 1, wherein automatically determining, if the resultant electrical waveforms are adequate for ongoing monitoring includes the results of further stimulation.

9. The method of claim 1, further comprising determining if the resultant electrical waveforms might be improved by a change in a stimulation frequency, intensity, or duration.

10. The method of claim 1, wherein integrating the combined available information from the electrical waveforms is performed by an algorithm or by an artificial intelligence classifier.

11. The method of claim 1, further comprising alerting a user to the detected change using at least one of:
a notification;
an alert;
a communication;
an indication; or
an alarm.

12. The method of claim 1, further comprising displaying information based on the resultant electrical waveforms on at least one display.

13. The method of claim 1, further comprising receiving a user input regarding an accuracy of the resultant electrical waveforms.

14. The method of claim 1, wherein the further stimulating step is due to user intervention, a predetermined frequency or sequence, or in response to a detected change in the electrical signals.

15. A method of identifying neural dysfunction and anesthetic effect using ear and forehead electrodes in a surgical patient, the method comprising:
stimulating at least one of one or more peripheral nerves in one or more extremity and one or more ears with sound;
recording resultant electrical waveforms generated by brainstem and cerebrum of the surgical patient in response to the stimulations using a recording electrode in one ear with a reference electrode in the other ear or over one or more areas of the forehead;
recording spontaneous electrical waveforms from the cerebrum using a recording electrode in one ear with a reference electrode in the other ear or over one or more areas of the forehead;
detecting, by a computing device, by various means, a change in the resultant electrical waveforms;
analyzing, by the computing device, by various means, the detected change in the resultant electrical waveforms to automatically identify the neural dysfunction, the neural dysfunction comprising an immediate or potential injury to one or more nerve structures due to stress associated with a surgical occurrence during a surgery;
analyzing, by the computing device, by various means, the detected change in the resultant electrical waveforms to automatically identify changes in anesthesia during a surgery;
analyzing, by the computing device, by various means, the detected change in the resultant electrical waveforms to automatically identify one or more effects of anesthesia on all of the resultant electrical waveforms;
and forwarding data to a display, the data comprising at least one of the electrical waveforms and an anatomical or functional diagram that depicts a location or degree of the detected change.
